SEQUENCE LISTING (8 sequences)

SEQ ID NO: 1 - DNA, 1572 bp, Homo sapiens
  misc_feature: H399W mutant hKv1.3 coding sequence
atgaccgtggtgcccggggaccacctgctggagccggaggtggccgatggtggaggggccccgcctcaaggcggctgtggcggcggcggctgcgaccgctacgagccgctgccgccctcactgccggccgcgggcgagcaggactgctgcggggagcgcgtggtcatcaacatctccgggctgcgcttcgagacgcagctgaagaccctttgccagttccccgagacgctgctgggcgaccccaagcggcgcatgaggtacttcgacccgctccgcaacgagtacttcttcgaccgcaaccggcccagcttcgacgccatcctctactactatcagtccgggggccgcatccgccggccggtcaacgtgcccatcgacattttctccgaggagatccgcttctaccagctgggcgaggaggccatggagaagttccgcgaggacgagggcttcctgcgggaggaggagcggcccttgccccgccgcgacttccagcgccaggtgtggctgctcttcgagtaccccgagagctccgggccggcccggggcatcgccatcgtgtccgtgctggtcatcctcatctccattgtcatcttctgcctggagacgctgccggagttccgcgacgagaaggactaccccgcctcgacgtcgcaggactcattcgaagcagccggcaacagcacgtcggggtcccgcgcaggagcctccagcttctccgatcccttcttcgtggtggagacgctgtgcatcatctggttctccttcgaactgctggtgcggttcttcgcttgtcctagcaaagccaccttctcgcgaaacatcatgaacctgatcgacattgtggccatcattccttattttatcactctgggtaccgagctggccgaacgacagggcaatggacagcaggccatgtctctggccatcctgagggtcatccgcctggtaagggtcttccgcatcttcaagctgtcgcgccactccaaggggctgcagatcctcgggcaaacgctgaaggcgtccatgcgggagctgggattgctcatcttcttcctctttattggggtcatccttttctccagcgcggtctactttgccgaggcagacgaccccacttcaggtttcagcagcatcccggatgccttctggtgggcagtggtaaccatgacaacagtgggttacggcgatatgtggccagtgaccatagggggcaagattgtgggatctctctgtgccatcgccggtgtcttgaccatcgcattgccagttcccgtgattgtttccaacttcaattacttctaccaccgggagacagaaggggaagagcaatcccagtacatgcacgtgggaagttgccagcacctctcctcttcagccgaggagctccgaaaagcaaggagtaactcgactctgagtaagtcggagtatatggtgatcgaagaggggggtatgaaccatagcgctttcccccagacccctttcaaaacgggcaattccactgccacctgcaccacgaacaataatcccaactcttgtgtcaacatcaaaaagatattcaccgatgtttaa

SEQ ID NO: 2 - protein, 523 aa, Homo sapiens
  misc_feature: H399W mutant hKv1.3
MTVVPGDHLLEPEVADGGGAPPQGGCGGGGCDRYEPLPPSLPAAGEQDCCGERVVINISGLRFETQLKTLCQFPETLLGDPKRRMRYFDPLRNEYFFDRNRPSFDAILYYYQSGGRIRRPVNVPIDIFSEEIRFYQLGEEAMEKFREDEGFLREEERPLPRRDFQRQVWLLFEYPESSGPARGIAIVSVLVILISIVIFCLETLPEFRDEKDYPASTSQDSFEAAGNSTSGSRAGASSFSDPFFVVETLCIIWFSFELLVRFFACPSKATFSRNIMNLIDIVAIIPYFITLGTELAERQGNGQQAMSLAILRVIRLVRVFRIFKLSRHSKGLQILGQTLKASMRELGLLIFFLFIGVILFSSAVYFAEADDPTSGFSSIPDAFWWAVVTMTTVGYGDMWPVTIGGKIVGSLCAIAGVLTIALPVPVIVSNFNYFYHRETEGEEQSQYMHVGSCQHLSSSAEELRKARSNSTLSKSEYMVIEEGGMNHSAFPQTPFKTGNSTATCTTNNNPNSCVNIKKIFTDV

SEQ ID NO: 3 - DNA, 1572 bp, Homo sapiens
  misc_feature: Wild type hKv1.3  coding sequence
atgaccgtggtgcccggggaccacctgctggagccggaggtggccgatggtggaggggccccgcctcaaggcggctgtggcggcggcggctgcgaccgctacgagccgctgccgccctcactgccggccgcgggcgagcaggactgctgcggggagcgcgtggtcatcaacatctccgggctgcgcttcgagacgcagctgaagaccctttgccagttccccgagacgctgctgggcgaccccaagcggcgcatgaggtacttcgacccgctccgcaacgagtacttcttcgaccgcaaccggcccagcttcgacgccatcctctactactatcagtccgggggccgcatccgccggccggtcaacgtgcccatcgacattttctccgaggagatccgcttctaccagctgggcgaggaggccatggagaagttccgcgaggacgagggcttcctgcgggaggaggagcggcccttgccccgccgcgacttccagcgccaggtgtggctgctcttcgagtaccccgagagctccgggccggcccggggcatcgccatcgtgtccgtgctggtcatcctcatctccattgtcatcttctgcctggagacgctgccggagttccgcgacgagaaggactaccccgcctcgacgtcgcaggactcattcgaagcagccggcaacagcacgtcggggtcccgcgcaggagcctccagcttctccgatcccttcttcgtggtggagacgctgtgcatcatctggttctccttcaaactgctggtgcggttcttcgcttgtcctagcaaagccaccttctcgcgaaacatcatgaacctgatcgacattgtggccatcattccttattttatcactctgggtaccgagctggccgaacgacagggcaatggacagcaggccatgtctctggccatcctgagggtcatccgcctggtaagggtcttccgcatcttcaagctgtcgcgccactccaaggggctgcagatcctcgggcaaacgctgaaggcgtccatgcgggagctgggattgctcatcttcttcctctttattggggtcatccttttctccagcgcggtctactttgccgaggcagacgaccccacttcaggtttcagcagcatcccggatgccttctggtgggcagtggtaaccatgacaacagtgggttacggcgatatgcacccagtgaccatagggggcaagattgtgggatctctctgtgccatcgccggtgtcttgaccatcgcattgccagttcccgtgattgtttccaacttcaattacttctaccaccgggagacagaaggggaagagcaatcccagtacatgcacgtgggaagttgccagcacctctcctcttcagccgaggagctccgaaaagcaaggagtaactcgactctgagtaagtcggagtatatggtgatcgaagaggggggtatgaaccatagcgctttcccccagacccctttcaaaacgggcaattccactgccacctgcaccacgaacaataatcccaactcttgtgtcaacatcaaaaagatattcaccgatgtttaa

SEQ ID NO: 4 - protein, 523 aa, Homo sapiens [sequence omitted]

SEQ ID NO: 5 - DNA, 45 bp, Artificial sequence
  Single strand DNA oligonucleotide
ttcattggggtcatccttttctccagtgcagtctactttgctgag

SEQ ID NO: 6 - DNA, 51 bp, Artificial sequence
  Single strand DNA oligonucleotide
aagggatcccacaatcttgcctcctatggtcactggccacatatcaccata

SEQ ID NO: 7 - DNA, 27 bp, Artificial sequence
  Single strand DNA oligonucleotide
catagatctgaatcggagtgagtgccg

SEQ ID NO: 8 - DNA, 28 bp, Artificial sequence
  Single strand DNA oligonucleotide
atgaattccacacaatactgggcacaga

(19) United States
(12) Patent Application Publication
(10) Pub. No.: US 2010/0015105 A1
(43) Pub. Date: Jan. 21, 2010

(54) METHOD OF TREATING SCHIZOPHRENIA

(76) Inventors: Shimon Marom, Haifa (IL); Zoya Gluzman, Mizpe Tal-El-Doar-Na Oshrat (IL); Yair Feld, Haifa (IL)

Correspondence Address: MARTIN D. MOYNIHAN d/b/a PRTSI, INC., P.O. BOX 16446, ARLINGTON, VA, 22215, US

(21) Appl. No.: 12/448,020

(22) PCT Filed: Dec. 25, 2007
(86) PCT No.: PCT/IL2007/001606

§ 371 Date: Jun. 4, 2009

Related U.S. Application Data
(60) Provisional application No. 60/877,161, filed on Dec. 27, 2006.

(51) Int. Cl. A61K 35/12; A61P 25/18

(52) U.S. Cl. 424/93.7

(57) ABSTRACT

A method of treating schizophrenia in a subject in need thereof is provided. The method comprising, administering to the subject a therapeutically effective amount of cells expressing at least one exogenous polypeptide forming a connexin channel and/or a hyperpolarizing ion channel, thereby treating the schizophrenia in the subject.

### FIELD AND BACKGROUND OF THE INVENTION

The present invention, in some embodiments thereof, relates to methods of treating schizophrenia.

Schizophrenia dramatically affects the health and well-being of individuals who suffer from this mental disorder, which is among the most severe and difficult to treat. Individuals with schizophrenia (“schizophrenics”) can suffer from a myriad of symptoms and may require significant custodial care and continuous drug and/or behavior therapy, leading to substantial social and economic costs, even in the absence of hospitalization or institutionalization. Schizophrenia affects approximately 2 million Americans. The illness usually develops between adolescence and age 30 and is characterized by one or more positive symptoms (e.g., delusions and hallucinations) and/or negative symptoms (e.g., blunted emotions and lack of interest) and/or disorganized symptoms (e.g., confused thinking and speech or disorganized behavior and perception). Schizophrenics have been demonstrated in many studies to have degraded abilities at tasks requiring short-term verbal working memory, rapidly associated cognitive “prediction” or “expectation”, or ongoing attention/vigilance control. Schizophrenics who have auditory hallucinations (which describes the majority of afflicted individuals) also have a strongly correlated degradation in their speech reception abilities. Schizophrenics also have social and functional skill deficits, e.g., deficits and confusion in identifying the moods or reactions of others, in determining what for them is a socially correct course of action and in identifying the sources of current and past actions or events. Schizophrenia is a chronic disorder and most patients require constant treatment to alleviate or decrease the incidence of psychotic episodes. The causes of schizophrenia are largely unknown. Although it is believed to have a genetic component, environmental factors appear to influence the onset and severity of the disease. The aggregation of schizophrenia in families, the evidence from twin and adoption studies, and the lack of variation in incidence worldwide, indicate that schizophrenia is primarily a genetic condition, although environmental risk factors are also involved at some level as necessary, sufficient, or interactive causes.

A number of studies aiming at treating schizophrenia and bipolar conditions using gene therapy have been described e.g., U.S. Patent Application Nos. 20070015152 teaches downregulation of a number of genes using siRNA, 20060127933 teaches overexpression of VMAT-1 and 20020193581 teaches regulation of RGS4 expression.

PCT WO02/33111 teaches methods of in vivo modifying the electrophysiological function of excitable tissues. The methods include the step of implanting cells (a) capable of forming gap junctions with at least one cell of the excitable tissue; and (b) capable of forming a functional ion channel or transporter, wherein the functional ion channel or transporter is capable of modifying the electrophysiological function of the excitable tissue. PCT WO02/33111 envisages the treatment of neurological disorders.

PCT WO06/018836 teaches cells expressing the Kv1.3 ion channel or its mutant form H401W as well as connexin 36 for the treatment of neurodegenerative diseases, such as Parkinson's disease.

Neither PCT WO02/33111 nor PCT WO06/018836 teaches treating Schizophrenia.

### SUMMARY OF THE INVENTION

According to an aspect of some embodiments of the present invention there is provided a method of treating schizophrenia in a subject in need thereof, the method comprising, administering to the subject a therapeutically effective amount of cells expressing at least one exogenous polypeptide forming a connexin channel and/or a hyperpolarizing ion channel, thereby treating the schizophrenia in the subject.

According to an aspect of some embodiments of the present invention there is provided the administering is effected to the substantia nigra pars compacta (SNc) and/or ventral tegmental area (VTA) of the subject.

According to an aspect of some embodiments of the present invention there is provided the administering is effected using a delivery route selected from the group consisting of direct injection during a neurosurgery procedure and trans-catheter through neural arteries.

According to an aspect of some embodiments of the present invention there is provided the cells are selected from the group consisting of myoblasts, fibroblasts, microglia, oligodendrocytes, astroglia, mesenchymal stem cells and embryonic stem cells.

According to an aspect of some embodiments of the present invention there is provided the connexin channel is selected from the group consisting of connexin32, connexin36, connexin43 and connexin47.

According to an aspect of some embodiments of the present invention there is provided the hyperpolarizing ion channel is selected from the group consisting of a potassium channel and a chloride channel.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1a-h are bar graphs showing the effects of the different treatments on locomotor hyperactivity behavior as determined looking at the following parameters: duration, distance and velocity parameters in an open field assay. Effect of cell transplantation on locomotor activity following apomorphine dosing is shown. Total movement, velocity and percent of time moving, measured during 20 minutes following apomorphine administration. Animals were tested 15 (FIGS. 1a-d) and 22 days (FIGS. 1e-h) after implantation. Significant differences between group 2 and control (group 3) on day 22 P<0.05 were shown. The differences between groups 1 and 2 were significant on both tests P<0.05 or P<0.01. The effects of treatment on stereotypic movement (10 cm<) versus locomotive movement (<10 cm) are shown in FIGS. 1d and 1h.

FIGS. 2a-c are bar graphs showing the effect of cell transplantation on prepulse inhibition measuring a startle reaction to a startle-eliciting stimulus. Measurements of PPI baseline levels (gray) and apomorphine (black) treatment on PPI response in vehicle (control) and group 1 or group 2 implanted cells

FIG. 3 is a bar graph showing prepulse inhibition. Measurements of PPI base line levels as a response of 6 dB subthreshold prepulse stimulus (gray) and apomorphine (black) in vehicle (control) and group 1 or group 2 implanted cells

### DESCRIPTION OF SPECIFIC EMBODIMENTS OF THE INVENTION

The present invention, in some embodiments thereof, relates to methods of treating schizophrenia.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Whilst reducing some embodiments of the present invention to practice, the present inventor has surprisingly uncovered that cell implantation at specific sites of the brain can significantly alleviate symptoms of schizophrenia in a schizophrenia animal model and as such can be used as a valuable therapeutic tool in the treatment of the disease.

As shown in the Examples section which follows, implantation of cells in specific sites of the brain significantly alleviated behavioral symptoms of animals treated with apomorphine. It is suggested that cell transplantation can change the physiological properties of the substantia nigra pars compacta (SNc) and/or ventral tegmental area (VTA) effecting dopamine secretion and therefore behavioral outcome in Schizophrenia patients.

Thus, according to some embodiments of the present invention, there is provided a method of treating schizophrenia in a subject in need thereof. The method comprising administering to the subject a therapeutically effective amount of cells expressing at least one exogenous polypeptide forming a connexin channel and/or a hyperpolarizing ion channel, thereby treating the schizophrenia in the subject. capable

According to an embodiment of this aspect of the present invention, the cells are capable of electrical coupling with the native brain tissue and affect (i.e., down-regulate) dopamine secretion therefrom.

Electrical coupling is usually achieved by gap junctions or electrical synapses although other mechanisms are also envisaged (e.g., fusion of grafted cell with host cell, and ion concentration changes). Electrical coupling is typically tested by impaling cells with microelectrodes, injecting a current into one, and looking for a change in potential in the other.

As used herein the term “schizophrenia” refers to a psychiatric diagnosis that describes a mental illness characterized by impairments in the perception or expression of reality, most commonly manifesting as auditory hallucinations, paranoid or bizarre delusions or disorganized speech and thinking in the context of significant social or occupational dysfunction. Typical symptoms include, but are not limited to, hallucinations, delusions, disordered thinking, movement disorders, flat affect, social withdrawal, and cognitive deficits.

As used herein the term “treating” refers to inhibiting or arresting the development of schizophrenia and/or causing the reduction, remission, or regression in the disease or symptoms.

As used herein the phrase “subject in need thereof” refers to a mammal (e.g., human) of any sex or age that is suspected of having schizophrenia, diagnosed with, or predisposed to schizophrenia.

According to an exemplary embodiment of the present invention the cells are genetically modified with at least one exogenous polypeptide forming a connexin channel and/or a hyperpolarizing ion channel.

Examples of cells which can be used in accordance with the teachings of the present invention include, but are not limited to, fibroblasts, myoblasts, microglia, oligodendrocytes, astroglia, mesenchymal stem cells and embryonic stem cells.

As used herein the phrase “hyperpolarizing ion channel” refers to an ion channel producing a positive current and therefore reducing the membrane voltage. Examples of such channels include, but are not limited to potassium channels and chloride channels. A non-limiting list of potassium ion channels include, but are not limited to, Kv family, Kir family, HERG, Delayed rectifier, and chloride channels: ClC family: ClC1, ClC2, ClCN3, ClC4, ClC6. Specific examples of the Kv family channels include Kv1.3 (e.g., wild type hKv1.3 SEQ ID NO: 3 and 4, GenBank Accession No. AAC31761) and mutants thereof [e.g., human H399W (SEQ ID NOs1 and 2)] and its rat homolog H401W (see e.g., Kupper J, Bowlby R M, Marom S, Levitan B T. Intracellular and extracellular amino acids that influence C-type inactivation and its modulation in a voltage-dependent potassium channel. Eur J Physiol. 1995; 430: 1-11, which is fully incorporated herein by reference). Other potassium channels which can be used in accordance with some embodiments of the present invention are described in WO2007/069247 which is fully incorporated herein by reference. A non-limiting list of examples of chloride ion channels include, but are not limited to, the CIC family (e.g., ClC1, ClC2, ClCN3, ClC4, ClC6)

As used herein the term “connexin” refers to the family of gao junction proteins such as set forth in InterPro Number IPR000500.

Examples of connexin proteins which can be used in accordance with these embodiments of the present invention include, but are not limited to, connexin32, connexin36, connexin43 and connexin47.

Methods of genetically modifying cells are well known in the art. Thus for example, to express the exogenous connexin and/or hyperpolarizing ion channels in mammalian cells, a polynucleotide sequence encoding the above-described polypeptide(s) is preferably ligated into a nucleic acid construct suitable for mammalian cell expression. Such a nucleic acid construct includes a promoter sequence for directing transcription of the polynucleotide sequence in the cell in a constitutive or inducible manner.

Constitutive promoters suitable for use with the present invention are promoter sequences which are active under most environmental conditions and most types of cells such as the cytomegalovirus (CMV) and Rous sarcoma virus (RSV). Inducible promoters suitable for use with the present invention include for example the inducible promoter such as for the tetracycline-inducible promoter (Zabala M, et al., Cancer Res. 2004, 64(8): 2799-804).

The nucleic acid construct (also referred to herein as an “expression vector”) of the present invention includes additional sequences which render this vector suitable for replication and integration in prokaryotes, eukaryotes, or preferably both (e.g., shuttle vectors). In addition, a typical cloning vectors may also contain a transcription and translation initiation sequence, transcription and translation terminator and a polyadenylation signal.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of the polypeptides of the present invention since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of the present invention into stem cells. Such methods are generally described in Sambrook et al., Molecular Cloning: A Laboratory Manual, Cold Springs Harbor Laboratory; New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

The cells according to the present invention can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the preparation accountable for the biological effect, i.e. the cells of the present invention.

Hereinafter, the phrases “physiologically acceptable carrier” and “pharmaceutically acceptable carrier” are interchangeably used to refer to a carrier, such as, for example, a liposome, a virus, a micelle, or a protein, or a dilutent which do not cause significant irritation to an organism and do not abrogate the biological activity and properties of the active ingredient. An adjuvant is included under these phrases.

Herein the term “excipient” refers to an inert substance added to a pharmaceutical composition to further facilitate administration of an active ingredient. Examples, without limitation, of excipients, include calcium carbonate, calcium phosphate, various sugars and types of starch, cellulose derivatives, gelatin, vegetable oils and polyethylene glycols.

Techniques for formulation and administration of compositions may be found in “Remington's Pharmaceutical Sciences,” Mack Publishing Co., Easton, Pa., latest edition, which is incorporated herein by reference.

Suitable routes of administration are preferably local rather than systemic, for example, via injection of the preparation directly into the excitable tissue region. Specific routes for local and systemic administration as well as transplantation techniques (e.g., CT guided) are listed in Examples 7-11 of the Examples section which follows.

For injection, the active ingredients of the invention may be formulated in aqueous solutions, preferably in physiologically compatible buffers such as Hank's solution, Ringer's solution, or physiological salt buffer.

Pharmaceutical compositions of the present invention may be manufactured by processes well known in the art, e.g., by means of conventional mixing, dissolving, granulating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically.

Pharmaceutical compositions suitable for use in context of the present invention include compositions wherein the active ingredients are contained in an amount effective to achieve the intended purpose.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer of the active ingredient. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed. (See e.g., Fingl, et al., 1975, in “The Pharmacological Basis of Therapeutics”, Ch. 1 p. 1).

In general, schizophrenia animal models can be divided in three categories, i.e. models that investigate behaviours in animals that are disturbed in schizophrenic patients (e.g. prepulse inhibition of the acoustic startle response and latent inhibition), pharmacological models, and experimentally induced brain pathology e.g. brain lesion models. Methods of generating such models and use of same are described in Bachevalier, J. (1994) Medial temporal lobe structures and autism, a review of clinical and experimental findings. Neuropsychologia 32, 627-648; R. Joober et al. Genetic of schizophrenia: from animal models to clinical studies. J. Psychiatry Neurosci. 2003; 27 (5): 336-47; Lipska, B. K., Jaskiw, G. E., Weinberger, D. R., 1993, Postpuberal emergence of hyperresponsiveness to stress and to amphetamine after neonatal hippocampal damage, a potential animal model for schizophrenia. Neuropsychopharmacol. 122, 35-43; Weinberger, R. R. (1987) Implications of normal brain development for the pathogenesis of schizophrenia. Arch. Gen. Psychiatry 44: 660-669; Wolterink G., Daenen, E. W. P. M., Dubbeldam, S., Gerrits, M. A. F. M., Van Rijn, R., Kruse, C. G., Van der Heijden, J., Van Ree, J. M. (2001) Early amygdala damage in the rat as model for neurodevelopmental psychopathological. Eur. Neuropsychopharmacol. 11, 51-59; and Daenen E. W. P. M., Wolterink G., Gerrits M. A. F. M., Van Ree J. M. (2002) Amygdala or ventral hippocampal lesions at two early stages of life differentially affect open filed behaviour later in life: an animal model of neurodevelopmental psychopathological disorders. Behavioral Brain Research 131: 67-78, each of which is fully incorporated herein by reference.

The data obtained from such animal studies can be used in formulating a range of dosage for use in human. The dosage may vary depending upon the dosage form employed and the route of administration utilized. The exact formulation, route of administration and dosage can be chosen by the individual physician in view of the patient's condition, (see e.g., Fingl, et al., 1975, in “The Pharmacological Basis of Therapeutics”, Ch. 1 p. 1). For example, Parkinson's patient can be monitored symptomatically for improved motor functions indicating positive response to treatment.

The amount of a composition to be administered will, of course, be dependent on the individual being treated, the severity of the affliction, the manner of administration, the judgment of the prescribing physician, etc. The dosage and timing of administration will be responsive to a careful and continuous monitoring of the individual changing condition. For example, a treated patient will be administered with an amount of cells which is sufficient to alleviate the symptoms of the disease, based on the monitoring indications.

The cells of the present invention may be co-administered with therapeutic agents useful in treating schizophrenia, such as antipsychotic drugs e.g., clozapine (Clozaril), risperidone (Risperdal), olanzapine (Zyprexa), quetiapine (Seroquel), ziprasidone (Geodon) and aripiprazole (Abilify). Alternatively, or additionally a non-drug treatment me be employed such as individual therapy and family therapy.

Following transplantation, the cells of the present invention preferably survive in the diseased area for a period of time (e.g. at least 6 months), such that a therapeutic effect is observed.

Implantation of cells can be effected by, for example, a syringe and needle adapted or fabricated for cell implantation, by a catheter drug delivery system (see for example, U.S. Pat. No. 6,102,887) or by standard neurosurgical methods.

The cells of the present invention can be administered to the treated subject using a variety of transplantation approaches, the nature of which depends on the site of implantation.

The term or phrase “administration”, “transplantation”, “cell replacement”, “implantation” or “grafting” are used interchangeably herein and refer to the introduction of the cells of the present invention to target tissue. The cells can be derived from the recipient (autologous) or from a non-autologous source (i.e., syngeneic, allogeneic or xenogeneic donor).

The cells are preferably grafted into at least one of SNc and VTA.

Conditions for successful transplantation include: (i) viability of the implant; (ii) retention of the graft at the site of transplantation; and (iii) minimum amount of pathological reaction at the site of transplantation. Methods for transplanting various nerve tissues, into host brains have been described in: “Neural grafting in the mammalian CNS”, Bjorklund and Stenevi, eds. (1985); Freed et al., 2001; Olanow et al., 2003). These procedures include intraparenchymal transplantation, i.e. within the host brain (as compared to outside the brain or extraparenchymal transplantation) achieved by injection or deposition of tissue within the host brain so as to be opposed to the brain parenchyma at the time of transplantation.

Since non-autologous cells are likely to induce an immune reaction when administered to the body several approaches have been developed to reduce the likelihood of rejection of non-autologous cells. These include either suppressing the recipient immune system or encapsulating the non-autologous cells in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol Biotechnol. 2001, 17: 249-60, and Lu M Z, et al., A novel cell encapsulation method using photosensitive poly(allylamine alpha-cyanocinnamylideneacetate). J Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Technol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002; 13:783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

Examples of immunosuppressive agents include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE.sup.R), etanercept, TNF.alpha. blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors and tramadol.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find support in the following examples.

### Examples

Reference is now made to the following examples, which together with the above descriptions illustrate some embodiments of the invention in a non limiting fashion.

Generally, the nomenclature used herein and the laboratory procedures utilized in the present invention include molecular, biochemical, cellular and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); all of which are incorporated by reference as if fully set forth herein. Other general references are provided throughout this document. The procedures therein are believed to be well known in the art and are provided for the convenience of the reader. All the information contained therein is incorporated herein by reference.

### Experimental Procedures

Animals—Male Fischer rats were used. The minimum and maximum weights of the group did not exceed ±20% of group mean weight of approximately 270-300 at study initiation. Acclimation period lasted a minimum of 5 days. Animal handling was according to the NIH and the Association for Assessment and Accreditation of Animal Care (AAALAC). Animals were housed in olyethylene cages (4/cage) measuring 25×30×15 cm with stainless steel top grill having facilities for pelleted food and drinking water in glass bottle; bedding; steam sterilized clean puddy husk (Harlan, Sani-chip catalog number 2018SC+F) was used and bedding material was changed along with the cage at least twice a week. Animals were housed under standard laboratory conditions, air conditioned and filtered (HEPA F6/6) with adequate fresh air supply. Animals were kept in a climate controlled environment. Temperature range was between 20-24° C. and relative humidity (RH) was between 30-70% with 12 hours of light and 12 hours dark cycle.

Diet—Animals were fed ad libitum a commercial rodent diet (Teklad Certified Global 18% protein diet). Animals had free access to drinking water.

Acute Schizophrenia induction—Dosing with Apomorphine (Schizophrenia inducer) was effected at 0.5 mg/kg IP administration.

DNA constructs and mutagenesis—pEGFP-N1 was purchased from Clontech (Clontech™ catalog number 6085-1), rat connexin36-EGFP-N3 was kindly provided by Dr. Georg Zoidl (Zoidl G. et al, J Neurosci Res. 2002). Mutatgenesis of wild type Kv1.3 channel (rKv1.3:H401W) was done as follows:

The tryptophane to histidine exchange at position 401 was achieved by cloning the wild type rat KV1.3 into the pGEM cloning vector, containing the endogenous Acc1 restriction site and the designed BamH1 restriction site on both sides of the desired mutation location (position 401). The following primers were used to create the mutation: rKv3(41-3) +Acc1-5′ TTC ATT GGG GTC ATC CTT TTC TCC AGT GCA GTC TAC TTT GCT GAG 3′ (SEQ ID NO: 5); and rKv3(41-3) +BamH1-5′ AAG GGA TCC CAC AAT CTT GCC TCC TAT GGT CAC TGG CCA CAT ATC ACC ATA 3′ (SEQ ID NO: 6).

Using PCR reaction the mutated 174 bp fragment was obtained and ligated into a PCR cloning kit (Qiagen®; Cat. No:231122). The DNA from positive E. coli colonies was isolated and sequenced. The wild-type fragment was then exchanged with the mutated fragment using the Acc1 and BamH1 restriction sites. Positive colonies were again sequenced to confirm the presence of the mutation. The exchanged Kv1.3 gene (referred to as H401W-1) was amplified with the following primers, each containing the designed restriction sites: rKv3 (41-3) s+Bgl2-5′ CAT AGA TCT GAA TCG GAG TGA GTG CCG 3′ (SEQ ID NO: 7); and rKv3 (41-3) as +EcoR1-5′ ATG AAT TCC ACA CAA TAC TGG GCA CAG A 3′ (SEQ ID NO: 8). Finally, the 1.7 kb mutated fragment was cloned into pIRES2:EGFP vector (Clontech™ catalog number 6029-1).

Primary rat dermal fibroblast isolation, culturing and transfection—Rat Dermal Fibroblasts (RDF) were isolated using collagenase digestion method (Wang H., et al, 2004). Small skin biopsies (˜5×3 cm² from neck or leg) were kept in a cold delivery medium (DMEM, Biological industries ltd., Israel) supplemented with 500 U/ml penicillin and 500 mg/ml streptomycin, 12.5 ug/ml Amphotericine, 250 ug/ml Gentamycin (Biological industries ltd., Israel) for 1-2 hours prior to cell harvesting. The skin was separated from underlying adipose tissue and washed three times with the fresh delivery medium. The tissue was minced into small pieces (2-3 mm³) and digested 3 hours in PBS solution containing 10 mg/ml Collagenase type 1 (4 ml/g tissue) at 37° C. under gently shaking conditions (50-55 rpm). Following incubation, the digest was filtrated through a 100 μm cell strainer (BD Falcon, Catalog Number 352360). The cell suspension was centrifuged at 250×g for 10 min and resuspended in the growth medium (DMEM supplemented with 10% Bovine Calf Serum (HyClone™), 2.5 ug/ml Amphotericine, 25 ug/ml Gentamycin, 2 mM Glutamine (Biological industries ltd., Israel)). The cells were seeded at a density of 5-10×10⁴ cells/cm² and grown till confluence, while the medium was changed every 3 days. The cells were expanded by passaging 1:3 until the required number of cells was obtained. RDFs at passage 5 were used in all experiments. In order to obtain H401W:GFP and Cx 36:GFP expressing RDF or only GFP expressing RDF as a control, we performed transfection using AMAXA nucleoporator according to the manufacturer's protocol. Expression of transgenes was confirmed 2 days after transfection using anti-GFP immunohistochemistry reaction. Three days after transfection the cells were trypsinized, washed with PBS and resuspended to the concentration 25,000 cells/μl in PBS supplemented with 1 mg/ml Glucose immediately prior to brain transplantation.

Immunohistochemistry—At the day of transfection, the genetically modified RDFs were seeded at a concentration of 2*10⁴ cells/well in Growth Medium on chamber slides (Lab-Tec Chamber Slide, cat #177399) and incubated for additional 48 hours. Cells were fixed with cold methanol at −20° C. for 10 minutes. After fixation the cells were permeabilized with 0.25% Triton in PBS for 15 min at room temperature followed by two PBS washes. Endogenous peroxidase activity was blocked by 10 min incubation with PO blocker (DAKO Co. Carpinteria™, CA, USA). The samples were blocked using CAS block (Zymed, Catalog Number 00-8120) for 20 min at room temperature to minimize unspecific background staining. Following blocking the cells were incubated with mouse anti-GFP antibody (SC9996 Santa Cruz) diluted 1:50 in the Abs diluent reagent (Zymed) for 2 hours at room temperature. The samples were washed 3 times with PBS and incubated with secondary anti-mouse antibodies (Mach 4 kit, Biocare Medical) for 30 min at room temperature. Slides were washed 3 times in PBS and enzymatic reaction with ready to use AEC (EnVision+ kit, DAKO Co. Carpinteria, CA, USA) was performed at room temperature for 10 min. The slides were mounted with VectaMount AQ mounting medium (H-5501, Vector, USA) and visualized under the microscope.

Cells implantation—4 μl of H401W:GFP/Cx36:GFP transfected cells, GFP modified cells (2.5*10⁴ cells/μl) in PBS supplemented with 1 mg/ml glucose, or Vehicle (saline supplemented with 1 mg/ml glucose) were implanted (bilaterally) at the following coordinates from Bregma:

SNc: Anterior-posterior axis (AP): −5.3, Left-right axis (LR): +/−2.4, Depth (DV): −7.9.

VTA: Anterior-posterior axis (AP): −6.0, Left-right axis (LR): +/−0.8, Depth (DV): −8.1.

The animal groups and experimental design are described in the following table. The experiment was done in the blind mode, and the animal test performer wasn't aware the type of material injected into the animals.

TABLE 1
Experimental design

| Group | | Apomorphine | PPI | |
|---|---|---|---|---|
| (n = 12) | Procedure | dosing regimen | assessment | Termination |
| Group 1 | Steriotaxic | Days 15, 22 | Days 27, 31 | Day 45 |
| | GFP cells | | (baseline) | Brain removal and |
| Group 2 | Steriotaxic | | Days 28 and | pathophysiological |
| | Cx36 + | | 32 (test) | assessment |
| | H399W | | | |
| | cells | | | |
| Control | Buffer | | | |

Body weight—Body weights were recorded before implantation and then weekly.

Termination and Necropsy—After completion of the second PPI tests animals were sacrificed and brains were harvested into formalin or glutaraldehyde solution (supplied by the sponsor) as follows: brains from six animals of group 1 and 6 animals from group 2 were fixed in glutaraldehyde, the rest of the animals—in formalin.

Behavioral Tests—

Behavioral tests were conducted 10 min following apomorphine administration for a period of 20 min. Observation lasted total of 30 min. Dosing with Apomorphine (Schizophrenia inducer) 1.5 mg/kg IP administration, was performed on days 15, 22 and 0.75 mg/kg IP at 36 days post cell implantation. Dosing Ketamine (noncompetetive NMDA antagonist) 16 and 30 mg/kg IP administration was performed a week after Apomorphine administration.

For efficacy assessment the following tests were done:

1. Locomotion test—open field (ethovison computerized analysis)

2. Inhibition of apomorphine stereotypy behavioral.

3. PPI study on day 28 and 32 (without), 29 and 43 with 0.5 mg/kg apomorphine compared to a baseline/training measurement done one day before (day 27 and 31).

Behavioral tests—Testing was performed in a blinded fashion.

I. Open Field: Apomorphine-Induced Locomotion Via Postsynaptic Dopaminergic Responses Rather than Direct Will be Tested in the Open Field.

The behavior is videotaped and scored by trained observers blind to experimental conditions. Locomotor activity is studied in an opaque open field (100 cm′100 cm′40 cm), the floor of which was marked with 20 cm′20 cm squares. The rats are habituated to the field of study for 10 min the day before testing. On the experimental day, the animals are placed in the center of the field and locomotor activity (number of lines crossed) scored during the 20 minutes observation.

II. PPI: The Baseline Session Used to Familiarize Rats with the Testing Procedure was Done One Day Before Test Trials and Consisted of:

20 trials: 10 min/animal

17 trials—120 dB (40 msec) Pulse alone—every 15 sec.

3 trials—2 dB (20 msec followed by (after 100 msec) 120 dB every 15 sec.

The trials with and without apomorphine were done in two consecutive days and consisted of:

20 trials: 20 min/animal

Five Different Trial Types

Pulse alone 40 msec 120 dB

Prepulse (20 msec) 3 dB (68 dB) thereafter 100 msec delay and 120 dB pulse (40 msec)

Prepulse (20 msec) 6 dB (71 dB) thereafter 100 msec delay and 120 dB pulse (40 msec)

Prepulse (20 msec) 12 dB (77 dB) thereafter 100 msec delay and 120 dB pulse (40 msec)

No stimulus—background only.

Second PPI test will be done two weeks after the first one with the same baseline kind of trials and the following trials with and without Apomorphine.

Each 40 trial test started and ended with 8 rounds of “Pulse alone 40 msec 120 dB”.

40 trials: 30 min/animal

### Five Different Trial Types (×8)

Pulse alone 40 msec 120 dB

Prepulse (20 msec) 3 dB (68 dB) thereafter 100 msec delay and 120 dB pulse (40 msec)

Prepulse (20 msec) 6 dB (71 dB) thereafter 100 msec delay and 120 dB pulse (40 msec)

Prepulse (20 msec) 12 dB (77 dB) thereafter 100 msec delay and 120 dB pulse (40 msec)

No stimulus—background only.

Termination—The animals were sacrificed on day 45 after cell implantation and brains were harvested for histopathological evaluation.

Statistical analysis—Numerical results were giver as means and standard deviations. Statistical analysis was carried out using the tow way ANOVA, repeated measure followed by Bonferroni post test. A probability of 5% (p≦0.05) was regarded as significant.

Results

The study was effected to confirm that cell implantation attenuates stereotypic behavior and locomotive activity following apomorphine administration. In addition, utilizing acoustic startle box, prepulse inhibition test (PPI) was included in order to assess potential antipsychotic effect.

Open Field

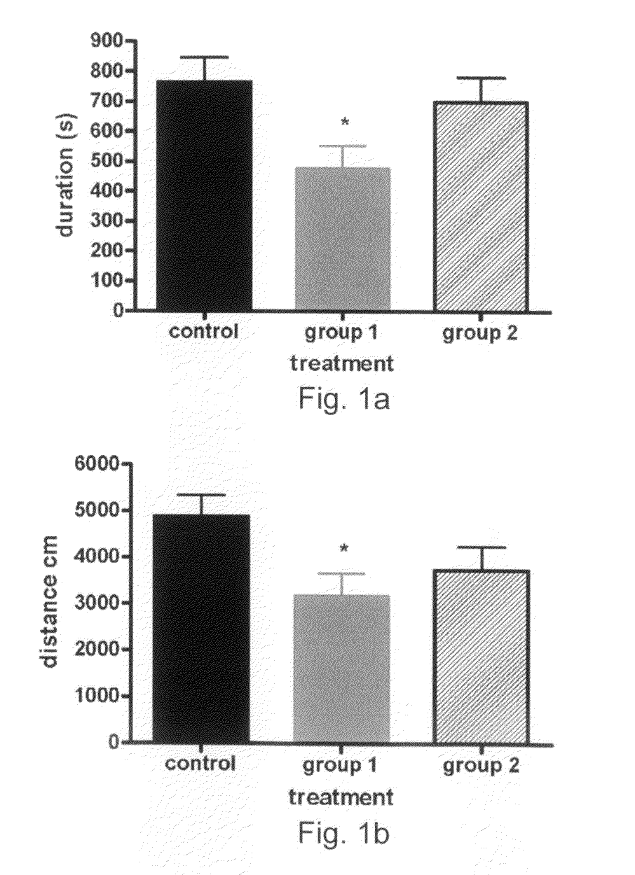

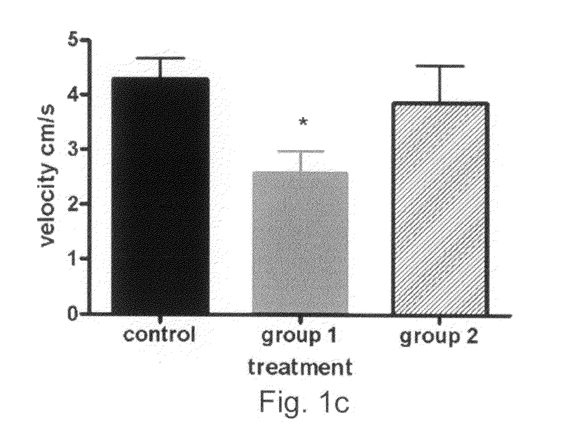

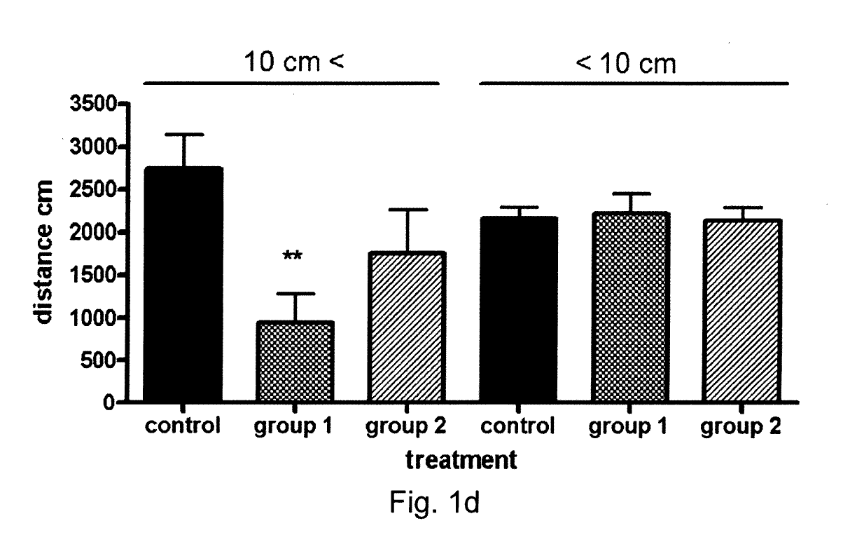

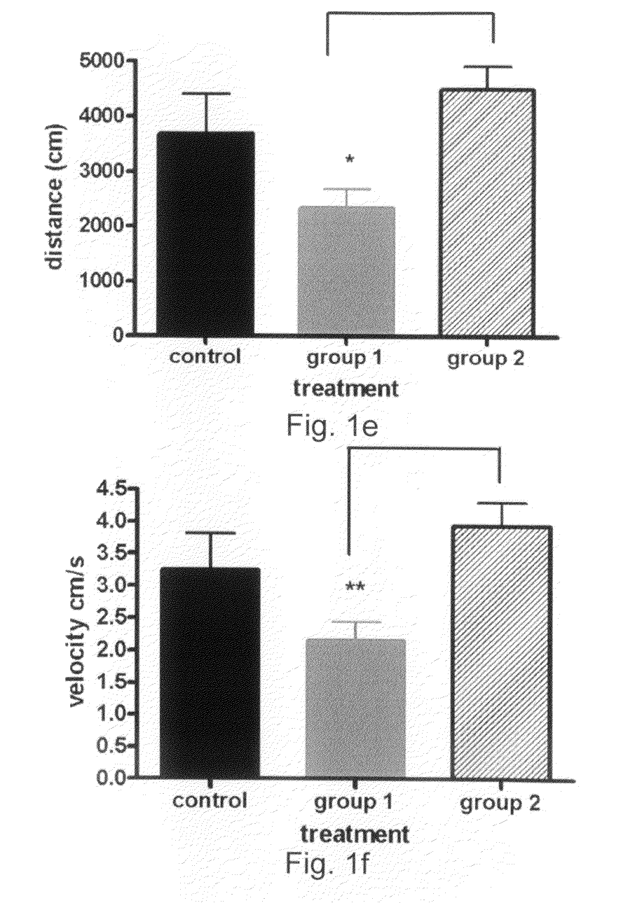

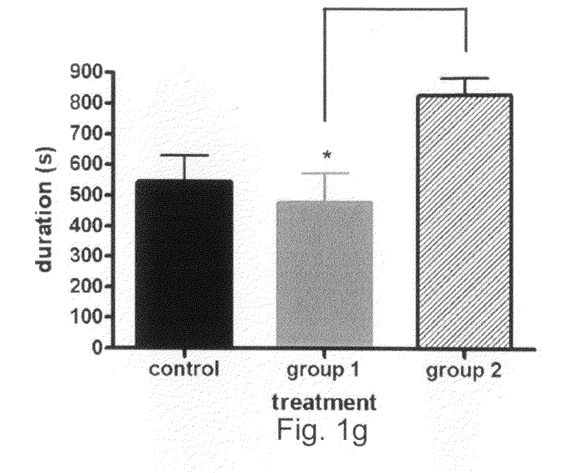

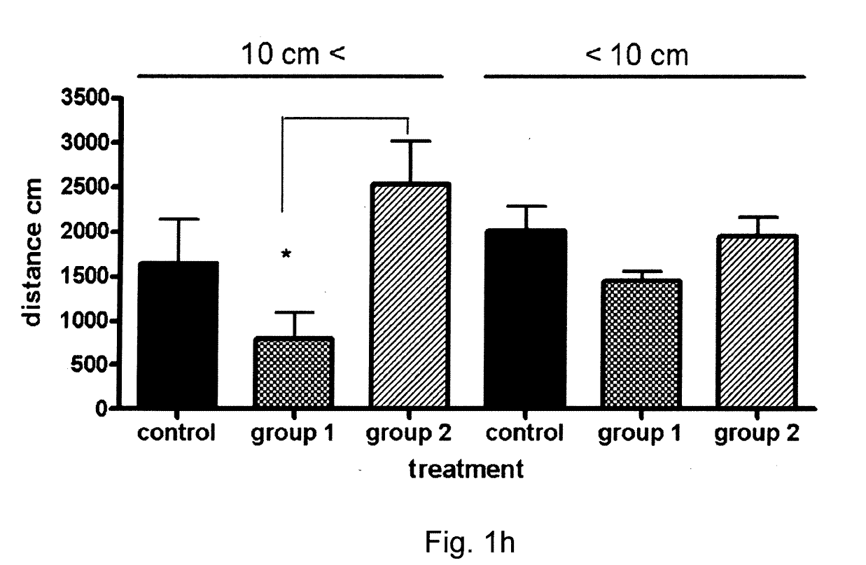

The open field test measured the response to apomorphine administration at 15 and 22 days following cells implantation. As can be seen from FIGS. 1a-c implantation of cells expressing the transgenes diminished response to apomorphine.

On day 15 cells implantation decreased locomotor hyperactivity as revealed by measurement of distance moved during the 20 min observation period (from 4895±444 control vehicle group to 3737±508; 3168±484 cm non-transfected cells vs transfected cells respectively, FIG. 1b). A significant difference could be demonstrated between the control group and the transfected cells group (p<0.05).

Reduction in the response to apomorphine administration could be demonstrated when analyzing two other parameters duration of movement (FIG. 1a) and velocity (FIG. 1c). In comparison with the distance measurement, a decrease in total movement duration could not be demonstrated between the control group and the non-transfected cells group (765±80 sec vs 700±81 sec; control vs non-transfected cells respectively). However, significant reduction was observed between the transfected cells group (group 1 treatment) and the control vehicle group (475±78 sec; p<0.05 in comparison with the control group).

In line with the duration findings, velocity (FIG. 1c) was also affected by cells implantation. Velocity significantly decreased in the transfected cells group, this, in comparison with the control vehicle group (p<0.05). Significant differences could not be observed between the non-transfected cells and the control vehicle groups (4.9±0.4, 3.9±0.0.7, 2.6±0.4 control, non-transfected, transfected cells respectively).

To ensure that the motor activity recorded is related to real locomotor movements in space and not due to repeated pixel changes within a limited space, the data were filtered, dividing movement to two general types: stereotypic behavior [distances below 10 cm] (see Flagstad et al., Neuropsychopharmacology, 2004, 29, 2052-2064) and above 10 cm which indicate locomotive movement.

As can be seen from FIG. 1d, on day 15, significant effect (p<0.01) on locomotive movement could be demonstrated when comparing between the control group and the transfected cells group (decreases from 2734±406 control group to 947±347 transfected cells group). Non-transfected cells did not affect locomotive movement to a significant manner (2394±787).

No effect on stereotypic-type movement could be demonstrated on day 15 (2160±134, 2188±148, 2219±237; control, non-transfected cells, transfected cells respectively).

As can be seen in FIGS. 1e-g, on day 22 a reduction in the three tested parameters was also demonstrated. However, unlike day 15, the observed effect on day 22 was significant only between the non-transfected and transfected groups and trend could be demonstrated between the transfected cells group and the vehicle group.

In line with day 15 observation, the effect was mainly confined to the locomotive type of movement (see Table 2 below and FIG. 1h). However, in the transfected cells group, small effect (although non-significant) could be demonstrated in stereotypic-type movement (from, ˜2000 cm to 1500 cm control or non-transfected cells groups vs transfected cells group respectively)

TABLE 2

| | Distance | | Velocity | Stereotypic | |
|---|---|---|---|---|---|
| Treatment | (cm) | Duration (s) | (cm/sec) | movement | Locomotion |
| | Day 15 | | | | |
| Control | 4895 ± 444 | 765 ± 80 | 4.9 ± 0.4 | 2160 ± 134 | 2734 ± 406 |
| Transfected cells | 3168 ± 484 (a) | 475 ± 78 (a) | 2.6 ± 0.4 (a) | 2219 ± 237 | 947 ± 347 (a) |
| Non transfected cells | 3737 ± 508 | 700 ± 81 | 3.9 ± 0.7 | 2141 ± 787 | 1596 ± 148 |
| | Day 22 | | | | |
| Control | 3659 ± 716 | 545 ± 85 | 3.2 ± 0.6 | 2018 ± 269 | 1641 ± 502 |
| Transfected cells | 2327 ± 352 (b) | 476 ± 95 (b) | 2.2 ± 0.3 (c) | 1450 ± 116 | 799 ± 295 (b) |
| Non transfected cells | 4485 ± 417 | 827 ± 56 | 3.9 ± 0.4 | 1953 ± 214 | 2532 ± 489 |

(a = p < 0.05 in comparison with the control group, b = p < 0.05 in comparison with the non transfected group, c = p < 0.01 in comparison with the non transfected group)

Over all, between days 15 and 22 there was a decrease in the response to apomorphine administration in the control and transfected cells groups (except from the duration parameter in the transfected group). In the non-transfected group, the response to apomorphine tends to increase in three tested parameters: distance, locomotive type movement and duration; decrease in stereotypic movement and no effect on velocity.

Prepulse Inhibition Test

It has been established that prepulse inhibition (PPI) is decreased in symptomatic schizophrenia patients. Essentially, prepulses do not diminish the startle reflex in schizophrenia patients to the extent that they do in people that don't have schizophrenia (Braff et al., 1978, Psychophysiology 15:339-343). This is thought to reflect an abnormality in sensorimotor gating of schizophrenia patients, an abnormality that is believed to account, at least in part, for their symptoms. Further evidence suggesting PPI abnormalities and schizophrenia symptoms are linked is that brain circuitry underlying both phenomena overlap considerably (Swerdlow et al., 2001, Psychopharmacology 156:194-215) and evidence that drugs that improve psychotic symptoms in schizophrenia patients also improve their PPI abnormalities (Kumari and Sharma, 2002, Psychopharmacology 162:97-101).

In this test, the attenuation produced by a low intensity stimulus presented just before the stratele stimulus was assessed. Since the degree of PPI is related to the intensity of prepulse more levels of prepulse intensity were included.

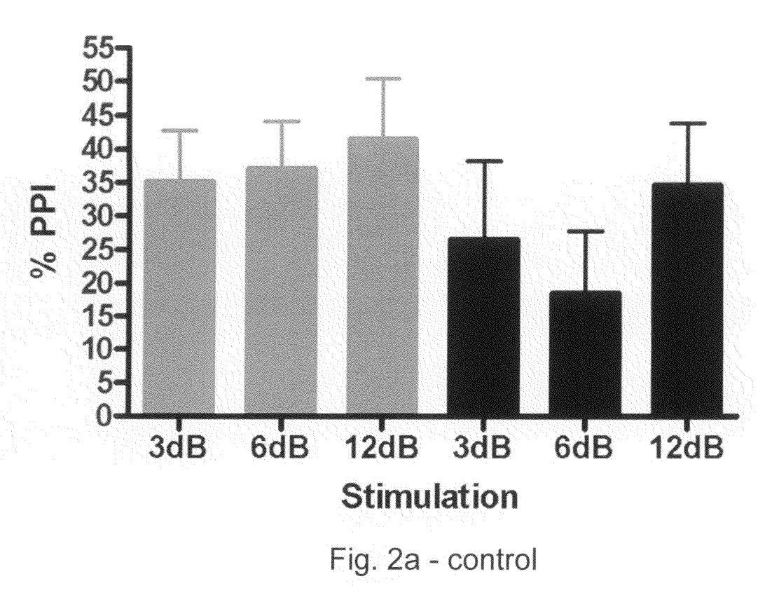

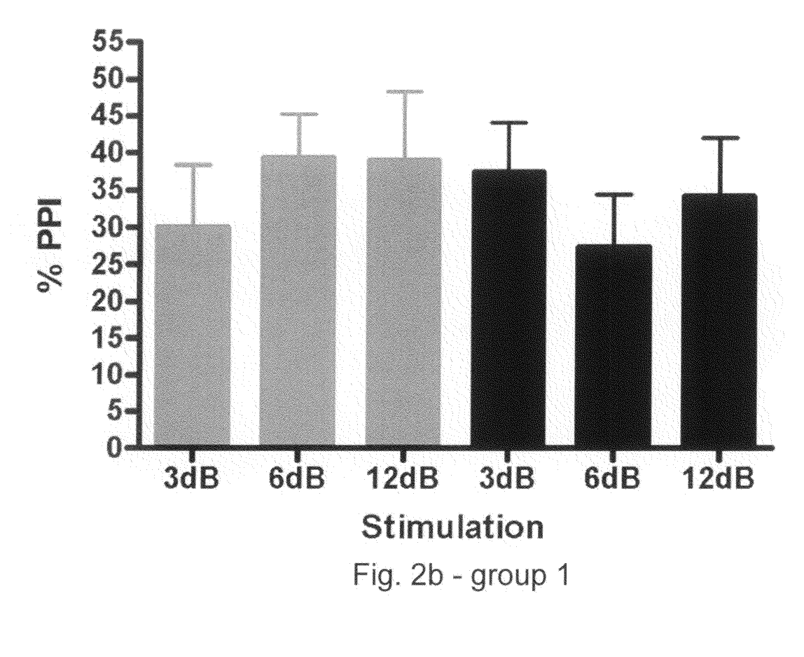

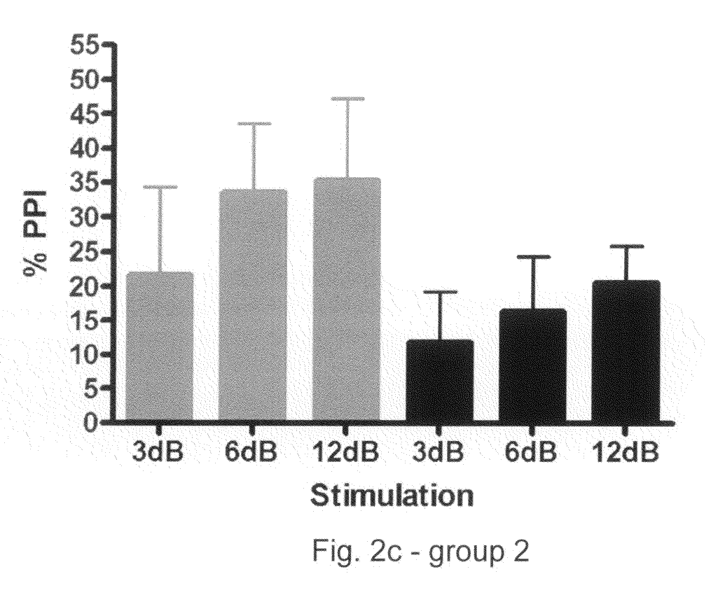

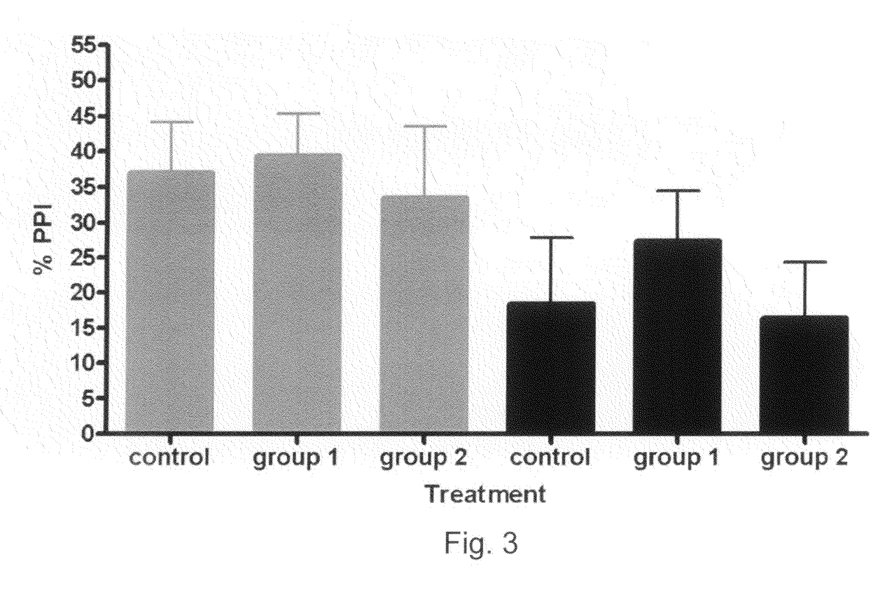

As can be seen from FIGS. 2a-c and FIG. 3 in all tested groups, PPI base line levels increased in a prepulse dependent manner (see Table 3 below) and no difference could be demonstrated between groups

TABLE 3
Effect of cell transplantation on base line PPI response and PPI values
following apomorphine dosing of treated and non-treated rats.

| Prepulse | Treatment group | | |
|---|---|---|---|
| intensity | control | group 1 | group 2 |
| Base line | | | |
| 3 dB | 35.2 ± 7.4 | 30 ± 8.3 | 21.7 ± 12.6 |
| 6 dB | 37.0 ± 7 | 39.4 ± 5.9 | 33.6 ± 10 |
| 12 dB | 41.4 ± 9 | 38.9 ± 9.2 | 35.2 ± 11 |
| After apomorphine | | | |
| 3 dB | 26.5. ± 11.2 | 37.4 ± 6.6 | 11.9 ± 7.4 |
| 6 dB | 18.4 ± 9.4 | 27.4 ± 7 | 16.4 ± 7.9 |
| 12 dB | 34.6 ± 9.2 | 34.2 ± 7.8 | 20.6 ± 5.3 |
| Δ response | | | |
| 3 dB | 9 | −7 | 10 |
| 6 dB | 19 | 12 | 17 |
| 12 dB | 7 | 4 | 15 |

To examine the effect of cells transplantation on apomorphine induced PPI disruption it was necessary to choose one stimulatory level where PPI base line values were similar.

Therefore, for evaluation of apomorphine disrupts PPI response the 6 dB prepulse level was selected. Similar base-line PPI values could be measured in this dose level (37.0±7, 39.4±5.9, 33.6±10, control, transfected cells, non-transfected cells groups respectively) and also, maximum disruption of PPI as a result of apomorphine administration.

When comparing groups, it appeared that the response to apomorphine was almost similar in the control and non-transfected cells groups. However, transfected cells, although not significantly, attenuated apomorphine induced PPI disruption. This finding is being supported by the observation that in all prepulse tested levels (3 dB and 12 Db), cells transfection had the same inhibitory effect on apomorphine.

Mortality and Body Weight

One rat (#12) from transfected cells group died 3 days after surgery. No effect on body weight or body weight gain could be demonstrated as a result of cells implantation.

Following bilateral cell transplantation into the VTA and SN there were no signs of adverse side effects in implanted animals.

### CONCLUSIONS

The results of this study are in a good agreement with the previous pilot study (U.S. Provisional Patent Application No. 60/877,161 which is hereby fully incorporated by reference).

The study results indicate that cells implantation into the VTA and SNc diminishes response to dopaminergic agonists. This attenuation is mainly on the locomotive type movement.

Overall, it was demonstrated that there is a difference between connexin 36 Kv1.3 expressing cells and control treatments.

Furthermore, on day 22 a significant difference could be demonstrated between the non transfected and the transfected cells in all tested parameters. This suggests that under this experimental condition, the non-transfected cells group can be considered as a control group.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

Braff D, Stone C, Callaway E, Geyer M, Glick I, Bali L (1978) Prestimulus effects on human startle reflex in normals and schizophrenics. Psychophysiology 15:339-343.
Kumari V, Sharma T (2002) Effects of typical and atypical antipsychotics on prepulse inhibition in schizophrenia: a critical evaluation of current evidence and directions for future research. Psychopharmacology (Berl) 162:97-101.
Swerdlow N R, Geyer M A, Braff D L (2001) Neural circuit regulation of prepulse inhibition of startle in the rat: current knowledge and future challenges. Psychopharmacology (Berl) 156:194-215.
Zoidl et al. Evidence for a role of the N-terminal domain in subcellular localization of the neuronal connexin36 (Cx36). J Neurosci Res. 2002 Aug. 15; 69(4):448-65.

What is claimed is:

1. A method of treating schizophrenia in a subject in need thereof, the method comprising, administering to the subject a therapeutically effective amount of cells expressing at least one exogenous polypeptide forming a connexin channel and/or a hyperpolarizing ion channel, thereby treating the schizophrenia in the subject.

2. The method of claim 1, wherein said administering is effected to the substantia nigra pars compacta (SNc) and/or ventral tegmental area (VTA) of the subject.

3. The method of claim 1, wherein said administering is effected using a delivery route selected from the group consisting of direct injection during a neurosurgery procedure and trans-catheter through neural arteries.

4. The method of claim 1, wherein said cells are selected from the group consisting of myoblasts, fibroblasts, microglia, oligodendrocytes, astroglia, mesenchymal stem cells and embryonic stem cells.

5. The method of claim 1, wherein said connexin channel is selected from the group consisting of connexin32, connexin36, connexin43 and connexin47.

6. The method of claim 1, wherein said hyperpolarizing ion channel is selected from the group consisting of a potassium channel and a chloride channel.
